(19) United States
(12) Patent Application Publication
(10) Pub. No.: US 2024/0087716 A1
(43) Pub. Date: Mar. 14, 2024

(54) COMPUTER-ASSISTED RECOMMENDATION OF INPATIENT OR OUTPATIENT CARE FOR SURGERY

(71) Applicant: Howmedica Osteonics Corp., Mahwah, NJ (US)

(72) Inventor: Jean Chaoui, Lyon (FR)

(21) Appl. No.: 18/273,176

(22) PCT Filed: Jan. 7, 2022
(86) PCT No.: PCT/US2022/011654

§ 371 Date: Jul. 19, 2023

Related U.S. Application Data
(60) Provisional application No. 63/139,508, filed on Jan. 20, 2021.

(51) Int. Cl. G16H 20/40; G16H 10/60; G16H 15/00; G16H 40/20; G16H 50/20

(52) U.S. Cl. CPC G16H 20/40; G16H 10/60; G16H 15/00; G16H 40/20; G16H 50/20

(57) ABSTRACT

A computer-implemented method comprises: obtaining, by a computing system, anatomic data for a patient and comorbidity data for the patient; generating, by the computing system, based on the anatomic data for the patient and the comorbidity data for the patient, a recommendation regarding whether the patient should undergo a surgery as an inpatient procedure or an outpatient procedure; and outputting, by the computing system, the recommendation.

This application claims priority to U.S. Provisional Patent Application 63/139,508, filed Jan. 20, 2021, the entire content of which is incorporated by reference.

### TECHNICAL FIELD

This disclosure relates to surgical procedure analysis and planning.

### BACKGROUND

In many cases, it is less expensive to perform a surgery as an outpatient procedure than as an inpatient procedure. This is because more resources are available to monitor and care for a patient during and after an inpatient procedure than an outpatient procedure. Because there may be fewer resources available when a surgery is performed as an outpatient procedure, it may be more challenging to provide appropriate care if complications arise from a surgery during or after an outpatient procedure.

### SUMMARY

This disclosure describes a variety of techniques for generating recommendations regarding whether a patient should undergo a surgery, such as a shoulder replacement surgery, as an inpatient procedure or an outpatient procedure. The techniques described in this disclosure may be used independently or in various combinations.

In one example, this disclosure describes a computer-implemented method comprising: obtaining, by a computing system, anatomic data for a patient and comorbidity data for the patient; generating, by the computing system, based on the anatomic data for the patient and the comorbidity data for the patient, a recommendation regarding whether the patient should undergo a surgery as an inpatient procedure or an outpatient procedure; and outputting, by the computing system, the recommendation.

In another example, this disclosure describes a computing system comprising: a memory configured to store anatomic data for a patient and comorbidity data for the patient; and one or more processing circuits configured to: generate, based on the anatomic data for the patient and the comorbidity data for the patient, a recommendation regarding whether the patient should undergo a surgery as inpatient procedure or an outpatient procedure; and output the recommendation.

In another example, this disclosure describes a computing system comprising: means for storing anatomic data for a patient and comorbidity data for the patient, means for generating, based on the anatomic data for the patient and the comorbidity data for the patient, a recommendation regarding whether the patient should undergo a surgery as an inpatient procedure or an outpatient procedure; and means for outputting the recommendation.

In another example, this disclosure describes a computer-readable medium having instructions stored thereon that, when executed, cause a computing system to perform any of the methods described in this disclosure.

The details of various examples of the disclosure are set forth in the accompanying drawings and the description below. Various features, objects, and advantages will be apparent from the description, drawings, and claims.

### BRIEF DESCRIPTION OF THE DRAWINGS

FIG. 1 is a block diagram of a surgical assistance system according to an example of this disclosure.

FIG. 2 is a flowchart illustrating an example operation of a surgical assistance system, in accordance with one or more techniques of this disclosure.

FIG. 3 is a flowchart illustrating an example supervised learning process to train a machine learning (ML) model to generate a recommendation regarding whether a patient should undergo a surgery as an inpatient procedure or an outpatient procedure, in accordance with one or more techniques of this disclosure.

### DETAILED DESCRIPTION

Joint replacement surgeries, such as shoulder replacement surgeries and ankle replacement surgeries, can be conducted as inpatient procedures or outpatient procedures. Conducting a joint replacement surgery as an outpatient procedure typically is less expensive than conducting the same joint replacement surgery as an inpatient procedure. However, if complications arise during the joint replacement surgery, a healthcare facility performing the inpatient procedure may be better equipped to handle the complications. Example types of complications may include stroke, cardiac events, anesthesiology reactions, excessive bleeding, bone fractures, infections, nerve or blood vessel damage, and so on.

In accordance with one or more techniques of this disclosure, a surgical planning system may generate a recommendation to a healthcare provider that indicates whether a patient can undergo a surgery as an outpatient procedure or should undergo the surgery as an inpatient procedure. For instance, the recommendation may indicate whether the patient can be treated at an outpatient healthcare facility or treated at an inpatient healthcare facility. The surgical planning system takes various types of data as input when making the recommendation. Example types of data that the machine learning system can take as input include patient demographic information, comorbidity information, and data regarding the patient's anatomy. Example types of patient demographic information may include age, sex, height, weight, and other information about the patient. Example types of comorbidity information may include smoking status, hypertension, chronic obstructive pulmonary disease, obesity, diabetes, steroid use, heart failure, and so on. Example types of data regarding the patient's anatomy may include bone density information, soft tissue quality, amounts of bone loss, and so on. The input may include 3-dimensional (3D) models of the joint or other information about the bones involved with the surgery.

The surgical planning system may apply one or more machine learning (ML) models to generate the recommendation whether the patient should undergo the surgery as an inpatient procedure or as an outpatient procedure. The ML models may be trained based on training data that maps input data to target output data. The target output data may be determined based on the recommendations of experienced surgeons regarding whether patients should undergo surgeries as inpatient procedures or outpatient procedures. Additionally, training data used for training the ML models may be selected from a larger set of data based on a set of factors, such as patient satisfaction with outcomes of the surgeries, occurrence of complications, occurrence of the need for reoperation, occurrence of readmission, cost, patient-reported outcome measurements (e.g., ranges of motion, achievement of mobility or activity goals, etc.), and length of stay following the surgery.

In some examples, the surgical planning system may also output an indication of an expected length of stay for the patient in a healthcare facility. In such examples, the training data used to train the ML models used by the surgical planning system may include target output data indicating actual lengths of stay of the patient. Example types of machine learning system that may be used include statistical analysis models (e.g., regression models), decision tree models, and artificial neural networks.

Certain examples of this disclosure are described with reference to the accompanying drawings, wherein like reference numerals denote like elements. It should be understood, however, that the accompanying drawings illustrate only the various implementations described herein and are not meant to limit the scope of various technologies described herein. The drawings show and describe various examples of this disclosure. In the following description, numerous details are set forth. However, it will be understood by those skilled in the art that the techniques of this disclosure may be practiced without these details and that numerous variations or modifications from the described examples may be possible.

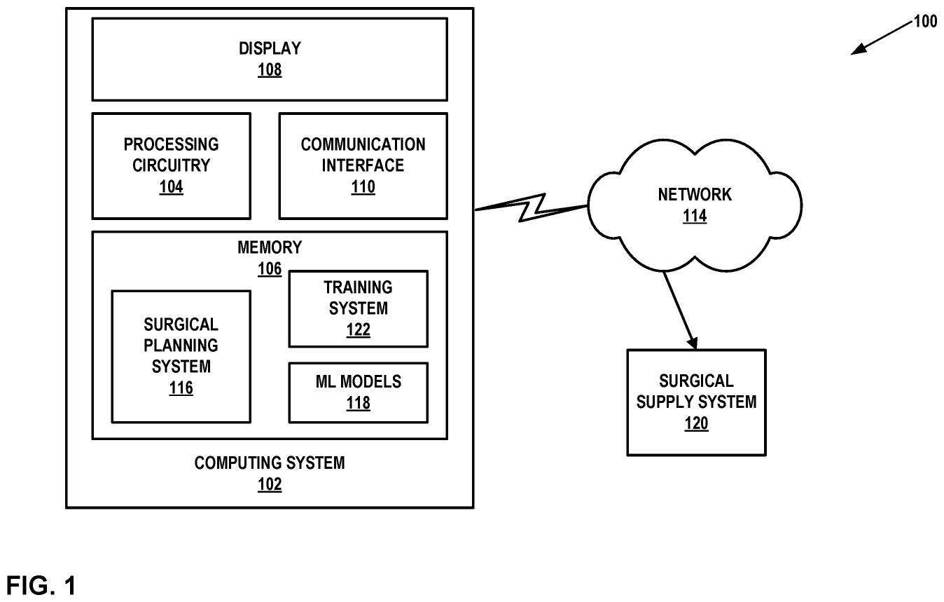

FIG. 1 is a block diagram illustrating an example surgical assistance system 100 that may be used to implement the techniques of this disclosure. FIG. 1 illustrates computing system 102, which is an example of a computing system configured to perform one or more example techniques described in this disclosure. Computing system 102 may include various types of computing devices, such as server computers, personal computers, smartphones, laptop computers, and other types of computing devices. Computing system 102 includes processing circuitry 104, memory 106, a display 108, and a communication interface 110. Display 108 may be optional, such as in examples where computing system 102 comprises a server computer.

Examples of processing circuitry 104 include one or more microprocessors, digital signal processors (DSPs), application specific integrated circuits (ASICs), field programmable gate arrays (FPGAs), hardware, or any combinations thereof. In general, processing circuitry 104 may be implemented as fixed-function circuits, programmable circuits, or a combination thereof. Fixed-function circuits are circuits that provide particular functionality and are preset on the operations that can be performed. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits.

Processing circuitry 104 may include arithmetic logic units (ALUs), elementary function units (EFUs), digital circuits, analog circuits, and/or programmable cores, formed from programmable circuits. In examples where the operations of processing circuitry 104 are performed using software executed by the programmable circuits, memory 106 may store the object code of the software that processing circuitry 104 receives and executes, or another memory within processing circuitry 104 (not shown) may store such instructions. Examples of the software include software designed for surgical planning. Processing circuitry 104 may perform the actions ascribed in this disclosure to computing system 102.

Memory 106 may store various types of data used by processing circuitry 104. For example, memory 106 may store anatomic data for a patient, comorbidity data for the patient, and other types of data. Memory 106 may be formed by any of a variety of memory devices, such as dynamic random access memory (DRAM), including synchronous DRAM (SDRAM), magnetoresistive RAM (MRAM), resistive RAM (RRAM), hard disk drives, optical discs, or other types of non-transitory computer-readable media. Examples of display 108 may include a liquid crystal display (LCD), a plasma display, an organic light emitting diode (OLED) display, or another type of display device.

Communication interface 110 allows computing system 102 to output data and instructions to and receive data and instructions from one or more devices via a network 114. Communication interface 110 may comprise hardware circuitry that enables computing system 102 to communicate (e.g., wirelessly or using wires) to other computing systems and devices, such as surgical supply system 120. Network 114 may include various types of communication networks including one or more wide-area networks, such as the Internet, local area networks, and so on. In some examples, network 114 may include wired and/or wireless communication links.

Furthermore, in the example of FIG. 1, memory 106 may include computer-readable instructions that, when executed by processing circuitry 104, cause computing system 102 to provide a surgical planning system 116. For ease of explanation, this disclosure may simply describe actions performed by computing system 102 when processing circuitry 104 executes instructions of a software system (e.g., surgical planning system 116, training system 122, etc.) as being performed by the software system (e.g., surgical planning system 116, training system 122, etc.).

One or more users may use surgical planning system 116 in a preoperative phase. For instance, surgical planning system 116 may help the one or more users generate a virtual surgical plan that may be customized to an anatomy of interest of a patient. The virtual surgical plan may include a 3-dimensional virtual model that corresponds to the anatomy of interest of the patient and 3-dimensional models of one or more prosthetic components matched to the patient to repair the anatomy of interest or selected to repair the anatomy of interest. The virtual surgical plan also may include a 3-dimensional virtual model of guidance information to guide a user in performing the surgical procedure, e.g., in preparing bone surfaces or tissue and placing implantable prosthetic hardware relative to such bone surfaces or tissue. In accordance with one or more techniques of this disclosure, the virtual surgical plan may also include information regarding trajectories for inserting screws, pins, or other items into a bone of the patient.

Certain techniques of this disclosure are described below with respect to a shoulder replacement surgery. Examples of shoulder replacement surgeries include, but are not limited to, reversed arthroplasty, augmented reverse arthroplasty, anatomic total shoulder arthroplasty, augmented total shoulder arthroplasty, and hemiarthroplasty. However, the techniques are not so limited, and surgical planning system 116 may be used for other types of surgeries. For example, the techniques of this disclosure may be performed with respect to lower extremity surgeries, (e.g., total ankle replacement surgeries, bunion surgeries, Charcot foot repair surgeries), knee surgeries, hip surgeries, and other types of surgeries.

As described herein, surgical planning system 116 may obtain anatomic data for a patient and comorbidity data for the patient. The anatomic data for the patient may include data regarding an anatomy of the patient. For example, the anatomic data for the patient may include data describing soft tissue of a surgical site, such as a shoulder joint of the patient. Data describing the soft tissue may include data regarding medical conditions affecting the soft tissue, describing lengths, levels of fatty infiltration, strengths, tension, or characteristics of muscles, tendons, ligaments, or other non-bone tissues. Other data describing the soft tissue may include data regarding locations, thickness, and/or quality of cartilage at the surgical site.

In some examples, the data describing the soft tissue includes biomechanical model data. The biomechanical model data characterizes a biomechanical model of the joint of the patient. For instance, the biomechanical model data may include data indicating at least one of: static stress of muscles on the bones of the joint, static stress of an orthopedic prosthesis on one or more of the bones of the joint, static stress of two orthopedic prostheses of the joint on each other, dynamic stress of muscles on the bones of the joint, dynamic stress of an orthopedic prosthesis on one or more of the bones of the joint, and dynamic stress of two orthopedic prostheses of the joint on each other. Surgical planning system 116 may obtain the biomechanical model based on data received as input by a user. The biomechanical model may include a skeletal model, a musculature model, a musculoskeletal model, a kinematics model, a dynamics model, a joint boundary model, and/or a topological model. However, this disclosure uses biomechanical model as a general term to represent all of them.

In some examples, the anatomic data for the patient may include data regarding the bones at the surgical site of the patient. For instance, in an example where the surgery is a shoulder replacement surgery, the anatomic data for the patient may include one or more of 3D anatomic measurements of a scapula and humerus of the patient. For example, 3D anatomic measurements of a scapula may include measurements of a size, inclination, radius, and/or version of the patient's glenoid fossa. 3D anatomic measurements of the humerus may include measurements of an inclination, version of the humerus, radius of a humeral head, and/or other measurements of the humerus. The anatomic data for the patient may also include data describing relationships between bones of the shoulder joint or other aspects of the bones, such as:
A critical shoulder angle (CSA). The CSA of the patient is an angle between a first line and a second line. The first line is a line between a most superior point on a border of a glenoid cavity of a scapula of the patient and a most inferior point on the border of the glenoid cavity of the scapula of the patient. The second line is a line between the most inferior point on the border of the glenoid cavity of the scapula of the patient and a most lateral point of an acromion of the scapula of the patient. In general, smaller CSAs are correlated with increased glenoid wear, which is a sign of cuff tear arthropathy.
A distance from a humeral head center to a glenoid center.
A distance from the acromion to the humeral head.
A scapula critical shoulder sagittal angle (i.e., an angle between the lines mentioned above for the CSA, as the lines would be seen from a sagittal plane of the patient).
A glenoid coracoid process angle (i.e., an angle between (1) a line from a tip of the coracoid process to a most inferior point on the border of the glenoid cavity of the scapula, and (2) a line from the most inferior point on the border of the glenoid cavity of the scapula to a most superior point on the border of the glenoid cavity of the scapula).
An infraglenoid tubrical angle (i.e., an angle between (1) a line extending from a most inferior point on the border of the glenoid cavity to a greater tuberosity of the humerus, and (2) a line extending from a most superior point on the border of the glenoid cavity to the most inferior point on the border of the glenoid cavity).
A scapula acromion index (i.e., the ratio between the distance from the plane of the glenoid cavity to the lateral edge of the acromion and the distance from the plane of the glenoid cavity to the lateral edge of the humeral head).
A humerus orientation (i.e., a value indicating an angle between (1) a line orthogonal to the center of the glenoid, and (2) a line orthogonal to the center of the humeral head, as viewed from directly superior to the patient).
A humerus direction (i.e., the polar angle between the projection of the glenohumeral axis and the anteroposterior axis of the glenoid).
A measure of humerus subluxation (i.e., the ratio of the posterior portion of the humeral head with regard to the scapular plane).
A humeral head best fit sphere (i.e., a measure (e.g., a root mean square) of conformance of the humeral head to a sphere).

In some examples, surgical planning system 116 may determine the anatomic data based on medical images of the patient, such as x-ray images, CT images, or other types of medical images. For instance, surgical planning system 116 may generate 3D models of the bones associated with the surgical site based on the medical images. Surgical planning system 116 may then use the 3D models to determine the anatomic data. In some examples, surgical planning system 116 may apply image segmentation techniques to medical images to identify parts of the medical images that correspond to bones or soft tissues. Surgical planning system 116 may use the segmented medical images to generate the models, identify features, or otherwise determine the anatomic data.

The anatomic data for the patient may also include data regarding the bone quality of the bones at the surgical site. For example, the anatomic data for the patient may include data regarding the presence and location of osteophytes at the surgical site. In some examples, the data regarding the bone quality of the bones at the surgical site may include data regarding the bone density one or more bones. The data regarding the bone density may be generated by performing a dual-energy x-ray absorptiometry (DXA) scan of the patient's humerus. In other examples, surgical planning system 116 may obtain data generated by performing a quantitative computed tomography (QCT) scan of the patient's humerus or scapula. Surgical planning system 116 may then analyze the obtained data to determine the Hounsfield units of the blocks in the regions of the bones, such as the diaphysis and metaphysis of the humerus.

In other examples of data regarding bone quality, surgical planning system 116 may obtain patient-specific values for one or more Giannotti measurements. A Giannotti measurement is a ratio between a thickness of the cortical bone of the humerus and a total diameter of the humeral diaphysis at a transverse plane passing through the humeral diaphysis. In other words, where the bone is a humerus, the one or more bone density metrics may include a value defined as (x−z)/z, wherein x is an inner diameter of the humerus on a plane located a specific distance inferior to a greater tuberosity of the humerus and z is an outer diameter of the humerus on the plane.

Different Giannotti measurements correspond to different thresholds used to create different binary masks of the bone. In other words, different Giannotti measurements may be generated when only considering parts of the bone having densities above the corresponding threshold. For example, different Giannotti measurements may be generated using thresholds of 900 Hounsfield Units (HU), 1000 HU, or 1100 HU. After applying the binary mask, surgical planning system 116 may measure the thickness of the cortical and the other parameters. Surgical planning system 116 may obtain the Giannotti measurements based on radiographs. CT scans, or other types of medical imaging data.

In another example of data regarding bone quality, surgical planning system 116 may obtain patient-specific values for one or more Tingart measurements. A Tingart measurement is a mean of a first value, a second value, a third value, and a fourth value. The first value is the lateral cortical thicknesses of the humeral diaphysis at a first level. The second value is the medial cortical thickness of the humeral diaphysis at the first level. The third value is the lateral cortical thickness of the humeral diaphysis at a second level. The fourth value is the medial cortical thicknesses of the humeral diaphysis at the second level. The first level is at a location on the humeral diaphysis where endosteal borders of the lateral and medial cortices of the humerus are first parallel to each other, as determined from the proximal tip of the humerus. The second level is defined as 20 mm distal from the first level. Surgical planning system 116 may obtain the Tingart measurements based on radiographs. Similar to Giannotti measurements, surgical planning system 116 may obtain Tingart measurements using different thresholds used to create different binary masks of the bone.

As mentioned above, surgical planning system 116 may obtain comorbidity data for the patient. The comorbidity data for the patient may include data regarding comorbidities of the patient. The comorbidities of the patient are health considerations of the patient other than the condition for which the patient is undergoing the surgery. Example comorbidities of the patient may include one or more of: history of smoking, hypertension, chronic obstructive pulmonary disease (COPD), obesity, diabetes, steroid use, heart failure, and so on. Surgical planning system 116 may obtain the comorbidity data for the patient from an electronic medical record of the patient, from indications of user input indicating the comorbidity information, and/or other sources.

In some examples, surgical planning system 116 may obtain information in addition to the anatomic data for the patient and the comorbidity data for the patient. For example, surgical planning system 116 may obtain data indicating an implant type of a prothesis to be implanted in the patient during the surgery. For instance, in an example where the surgery is a shoulder replacement surgery, the implant type may be one of a glenoid cup implant or a glenosphere implant for an anatomic shoulder replacement surgery or a reverse shoulder replacement surgery, respectively. In some examples, surgical planning system 116 may use one or more ML models to generate a recommendation of the implant type. In some examples, surgical planning system 116 may also obtain data indicating patient demographic information, such as the age, of the patient. Surgical planning system 116 may obtain the patient demographic information from an electronic medical record for the patient, indications of user input of the patient demographic information, and/or from other sources.

In some examples, surgical planning system 116 may obtain data regarding bone loss of the patient. For example, surgical planning system 116 may obtain data indicating premorbid shapes of one or more bones of the patient at the surgical site. The premorbid shape of a bone corresponds to a shape of the bone prior to onset of a condition, such as an injury, osteoarthritis, etc. that changed the shape of the bone. In some examples, surgical planning system 116 uses statistical shape modeling to generate 3D models of the premorbid shapes of the bones. In some examples, surgical planning system 116 may use medical images captured before onset of the condition to generate the 3D models of the premorbid shapes of the bones. Surgical planning system 116 may compare the 3D models of the premorbid shapes of the bones to 3D models of the current shapes of the bones to identify locations that are marked as bone in the 3D models of the premorbid shapes of the bones that are not marked as bone in the 3D models of the current shapes of the bones. The identified locations are areas of bone loss. The obtained data may include data regarding locations of the areas of bone loss, total volume of bone loss, volume of bone loss in particular regions, and/or other information regarding the areas of bone loss.

Additionally, surgical planning system 116 may generate a recommendation regarding whether the patient should undergo a surgery (e.g., shoulder replacement surgery) as an inpatient procedure or an outpatient procedure. Surgical planning system 116 may generate the recommendation based at least in part on the anatomic data for the patient, the comorbidity data for the patient, and/or other types of data (e.g., demographic data, implant type, bone loss data, etc.) obtained by surgical planning system 116. Surgical planning system 116 may apply one or more machine learning (ML) models 118 to generate the recommendation. For instance, surgical planning system 116 may apply one or more artificial neural networks, statistics-based ML systems (e.g., linear regression, logistic regression), decision tree models, support vector machines, naïve Bayesian models, k-nearest neighbors models, k-means clustering models, random forest models, or other types of ML models to generate the recommendation. In some examples, surgical planning system 116 may use dimensionality reduction algorithms and/or gradient boosting algorithms, such as a Gradient Boosting Machine algorithm, a XGBoost algorithm, a LightGBM algorithm, or a CatBoost algorithm.

As mentioned above, surgical planning system 116 may apply one or more ML models 118 to generate the recommendation regarding whether the patient should undergo the surgery as an inpatient procedure or an outpatient procedure. In some examples, surgical planning system 116 may use the same ML models 118 to generate the one or more other types of output information. For example, surgical planning system 116 may apply a ML model to the anatomic data for the patient and the comorbidity data for the patient to generate the recommendation. In this example, surgical planning system 116 may apply the same ML model to the anatomic data for the patient and the comorbidity data for the patient to generate the duration of stay estimate for the patient. In other examples, surgical planning system 116 may apply different ones of ML models 118 to generate different types of outputs.

Surgical planning system 116 may output the recommendation. For example, surgical planning system 116 may output the recommendation via display 108, or another device or component. In some examples, surgical planning system 116 outputs the recommendation in a surgical planning interface used by the surgeon while the surgeon is planning the surgery. In some examples, surgical planning system 116 outputs the recommendation to a computing system of a health insurance provider.

In some examples, surgical planning system 116 may generate other types of output information based on the anatomic data for the patient, comorbidity data for the patient, and/or other types of information regarding the patient, such as the other types of information obtained by surgical planning system 116 described elsewhere in this disclosure. For example, surgical planning system 116 may generate a duration of stay estimate for the patient, wherein the duration of stay estimate for the patient is an estimate of a length of time the patient will stay in a healthcare facility after undergoing the shoulder replacement surgery. Other types of data that may be generated by surgical planning system 116 may include an estimated patient satisfaction, an expected complication rate for the patient, an expected reoperation rate for the patient, an expected readmission rate for the patient, an expected cost of the surgery, and so on. Reoperation is the performance of another surgery to correct the earlier surgery.

In the example of FIG. 1, surgical assistance system 100 also includes a surgical supply system 120. Surgical supply system 120 may comprise a computer system that is configured to send surgical items to healthcare facilities, such as inpatient healthcare facilities and outpatient healthcare facilities. In some examples, surgical planning system 116 may output the recommendation to surgical supply system 120. Accordingly, surgical supply system 120 may automatically send packages containing surgical items associated with the surgery to an inpatient healthcare facility or outpatient healthcare facility based on the recommendation. For example, surgical supply system 120 may send a more complex set of surgical items if the recommendation is to perform an inpatient (or non-ambulatory surgery center) procedure than if the recommendation is to perform an outpatient (or ambulatory surgery center) procedure. For instance, in this example, if the recommendation is to perform an inpatient procedure, the set of surgical items may include a wider variety of implants or instruments that may help a surgeon handle a wider range of contingencies.

A training system 122 may perform a training process to train ML models 118. Surgical planning system 116 may train the ML models based on training data that maps the input data to target output data. The target output data may be determined based on the recommendations, e.g., inpatient or outpatient procedure recommendations, of experienced surgeons. Additionally, training data used for training the machine learning system may be selected from a larger set of data based on a set of factors, such as patient satisfaction, occurrence of complications, occurrence of reoperation, occurrence of readmission, cost, patient-reported outcome measurements, and length of stay. For example, the training data used for training the ML models may represent cases in which there was high patient satisfaction, no complications, no reoperation, etc.

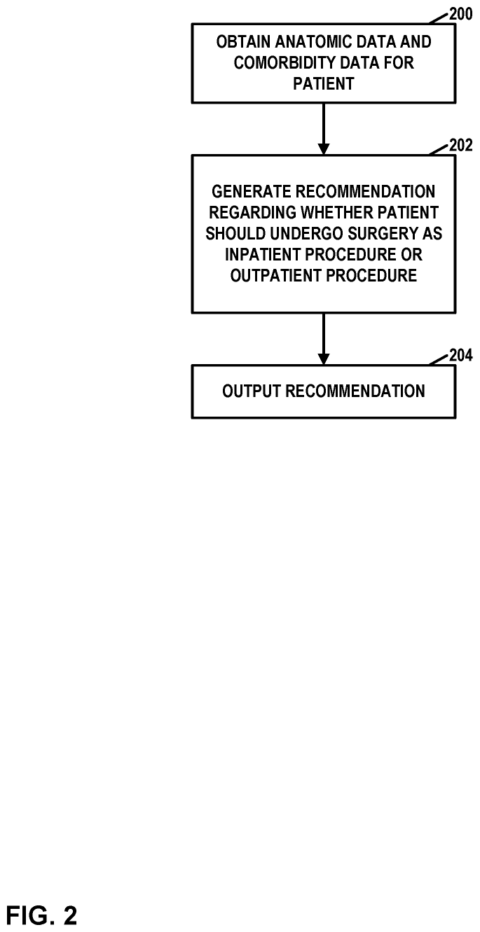

FIG. 2 is a flowchart illustrating an example operation of surgical planning system 116 in accordance with one or more techniques of this disclosure. The flowcharts of this disclosure are provided as examples. Other examples consistent with the techniques of this disclosure, the operations and processes shown in the flowcharts may include more, fewer, or different steps; or steps may be performed in different orders or in parallel.

In the example of FIG. 2, surgical planning system 116 may obtain anatomic data for a patient and comorbidity data for the patient (200). For example, surgical planning system 116 may generate or retrieve the anatomic data, comorbidity data, and/or other types of data regarding the patient, e.g., as described above.

Surgical planning system 116 may generate, based on the anatomic data for the patient and the comorbidity data for the patient, a recommendation regarding whether the patient should undergo a surgery as an inpatient procedure or an outpatient procedure (202). An inpatient procedure may be performed in a setting that is equipped to provide close monitoring of a patient during and after the surgery. Such a setting may include a bed, a room, an overnight stay for the patient, an attending healthcare professional, and other resources for close monitoring of the patient. An outpatient procedure may be performed in a setting, such as an ambulatory surgery center, in which the patient is expected to leave a healthcare facility on the same day as the surgery. In general, when a surgery is performed as an outpatient procedure, the patient's recovery is generally unsupervised. In some examples, outpatient procedures may be performed at healthcare facilities that provides healthcare without hospital admission. In general, outpatient healthcare facilities are less equipped to provide close monitoring of the patient during and after the surgery. In some examples, the recommendation may specify that the patient should undergo the surgery as an inpatient procedure or an outpatient procedure. As described elsewhere in this disclosure, surgical planning system 116 may use one or more ML models 118 to generate the recommendation.

Furthermore, in the example of FIG. 2, surgical planning system 116 may output the recommendation (204). In some examples, surgical planning system 116 may output the recommendation to surgical supply system 120, e.g., in response to receiving an indication that the surgeon accepts the recommendation. Surgical supply system 120 may automatically ship surgical items associated with the surgery to an inpatient healthcare facility if the recommendation indicates that the patient should undergo the surgery as an inpatient procedure or ship the surgical items associated with the surgery to an outpatient healthcare facility if the recommendation indicates that the patient should undergo the surgery as an outpatient procedure. Such surgical items may include implants (e.g., patient-matched implants, generic implants), surgical tools, pins, plates, screws, etc. In some examples, surgical planning system 116 may output the recommendation to display 108. For instance, display 108 may show the recommendation in a 2-dimensional graphical user interface (GUI).

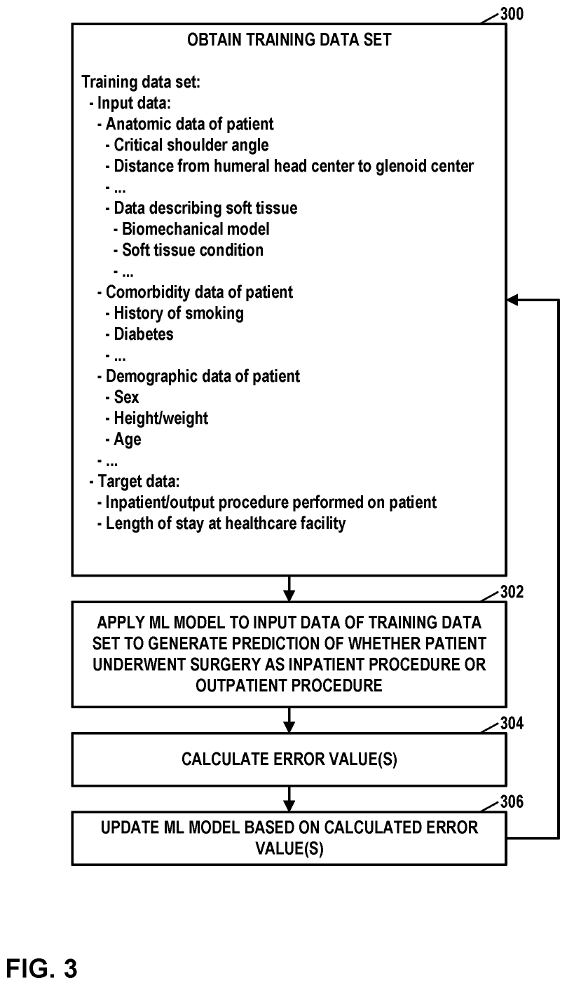

FIG. 3 is a flowchart illustrating an example supervised learning process to train a ML model to generate a recommendation regarding whether a patient should undergo a surgery as an inpatient procedure or an outpatient procedure, in accordance with one or more techniques of this disclosure. The ML model discussed with respect to FIG. 3 may be one of ML models 118 (FIG. 1).

In the example of FIG. 3, training system 122 may obtain a training data set (300). For example, training system 122 may obtain the training data set from a data store. In some examples, training system 122 may obtain the training data set from a user interface. In some examples, the training data set may be selected in various ways, depending on how the ML model is to be trained. For example, the training data sets may be limited to those related to surgeries performed by specific surgeons or surgical groups, surgeons performed in specific countries or regions, or based on other criteria. The training data sets may be selected such that the training data sets are limited to cases in which the patients expressed satisfaction with outcomes of their surgeries, did not experience complications, did not need reoperation, were not readmitted, had satisfactory patient-reported outcome measurements, and so on. In some examples, each of the training data sets is based on surgeries performed by or recommended by expert surgeons. In some examples, the training data sets may be limited to specific healthcare systems, healthcare insurance networks, or healthcare facilities.

The training data set includes input data and target data. The input data includes data that is provided to the ML model as input. For instance, in examples where the ML model comprises an artificial neural network, each item of input data may correspond to a different input neuron of the artificial neural network. The input data may include anatomic data of a patient and comorbidity data of the patient. In the example of FIG. 3, the anatomic data includes a critical shoulder angle, a distance from the humeral head center to the glenoid center, data describing soft tissue (e.g., a biomechanical model, a soft tissue condition, etc.), and so on. In other examples, the anatomic data may include other information. Furthermore, in the example of FIG. 3, the comorbidity data includes history of smoking and diabetes. In some examples, the input data may include demographic data of the patient. Example types of demographic data for the patient may include sex, height, weight, age, and other information about the patient that may have a statistical effect on the recommendation of whether the patient should undergo the surgery as an inpatient procedure or an outpatient procedure.

The target data of the training data set may specify whether the patient underwent the surgery as an inpatient procedure or as an outpatient procedure. In some examples, the target data may include other types of data. For instance, in some examples, the target data may include a length of stay that the patient had following the surgery.

Furthermore, during the supervised learning process, training system 122 may apply the ML model to the input data of the training data set to generate a prediction of whether the patient of the training data set would undergo the surgery as an inpatient procedure or an outpatient procedure (302). For instance, in an example where the ML model is an artificial neural network, training system 122 may provide numerical representations of the input data to individual neurons of an input layer of the artificial neural network. In this example, training system 122 may perform a forward propagation pass through the artificial neural network to generate one or more output values. The output values include the prediction of whether the patient of the training data set would undergo the surgery as the inpatient procedure or the outpatient procedure. In some examples, the output values include a duration of stay estimate for the patient that indicates an estimated length of stay of the patient at a healthcare facility following the surgery.

Training system 122 may calculate one or more error values based on the output values of the ML model (304). The error values may indicate differences between the output values of the ML model and the target data of the training data set. For example, an error value may indicate whether the prediction of whether the patient of the training data set would undergo the surgery as the inpatient procedure or the outpatient procedure is the same as the target data indicating whether the patient actually underwent the surgery as the inpatient procedure or the outpatient procedure. In an example where the output values of the ML model include a length of stay, the error value may indicate a difference between the length of stay indicated by the output value and an actual length of stay as indicated by the target data. In other examples, training system 122 may train separate ML models (e.g., different ones of ML models 118) for generating the recommendation and the estimate of the length of stay. Training system 122 may train the ML model for the estimate of the length of stay in a similar or different process from the ML model for generating the recommendation.

Training system 122 may update the ML model based on the calculated error value(s) (306). For instance, in an example where the ML model includes an artificial neural network, training system 122 may use the error value(s) in a backpropagation process that updates weights associated with inputs to neurons of the artificial neural network. During the training process, training system 122 may repeat steps 300 through 306 multiple times. In some examples, after the initial training process is complete and surgical planning system 116 subsequently applies the ML model to generate a recommendation regarding whether a patient should undergo the surgery as the inpatient procedure or the outpatient procedure, training system 122 may generate a new training data set for the patient and the target data may indicate whether the patient actually underwent the surgery as the inpatient procedure or the outpatient procedure, regardless of the recommendation.

Although the example of FIG. 3 shows an example process for performing a supervised learning process to train the ML model, in other examples, training system 122 may perform an unsupervised learning process to train the ML model. For example, if the ML model is a K-means clustering model, each of the training data sets may be present at the beginning of the training process and may be converted into a vector of numerical values. Furthermore, in this example, when training the K-means clustering model, training system 122 may iteratively update positions of a first centroid and a second centroid. In this example, the first centroid corresponds to the inpatient procedure and the second centroid corresponds to the outpatient procedure. In this example, the training data set may not include the target data.

Thus, in the example of FIG. 3 and other examples, training system 122 may train the ML model based on a plurality of training data sets, where for each respective training data set of the training data sets, the respective training data set specifies anatomic data for a respective earlier patient and comorbidity data for the respective earlier patient, and the respective training data set specifies whether the respective earlier patient underwent the surgery as the inpatient procedure or the outpatient procedure. Generation of the training data sets may involve data collection and data preparation. Data collection may include obtaining data from preoperative planning systems and records of orders of surgical items, such as implants. Data preparation may include cleaning potential training data sets so that only potential training data sets with complete information are retained. Additionally, as part of data preparation, training system 122 may normalize data in the training data sets and convert data types to ensure consistency among the training data sets. Training system 122 may also randomize the training data sets to reduce the effect of the order in which the training data sets are collected or otherwise generated. Training system 122 may also detect relationships (e.g., correlations) between variables in training data sets that can cause bias. Based on such relationships, training system 122 may remove specific variables from the training data sets. When performing the training process itself, training system 122 may use the training data sets to iteratively update parameters of the ML model in order to improve the predictions generated by the ML model.

In some examples, to generate the training data sets, input data for training data sets (i.e., the data that would be provided to the ML model as input) regarding the same set of patients may be provided to a group of surgeons. Each of the surgeons may independently make a recommendation for each patient regarding whether to perform an inpatient procedure or an outpatient procedure for the patient. Then, for each of the patients, the majority recommendation for the patient may be selected as the target recommendation for the training data set.

The following is a non-limiting list of aspects in accordance with one or more techniques of this disclosure.

Aspect 1: A computer-implemented method includes obtaining, by a computing system, anatomic data for a patient and comorbidity data for the patient; generating, by the computing system, based on the anatomic data for the patient and the comorbidity data for the patient, a recommendation regarding whether the patient should undergo a surgery as an inpatient procedure or an outpatient procedure; and outputting, by the computing system, the recommendation.

Aspect 2: The method of aspect 1, wherein the recommendation is regarding whether the patient should undergo the surgery at an inpatient healthcare facility or an outpatient healthcare facility.

Aspect 3: The method of aspect 1, wherein generating the recommendation comprises generating, by the computing system, the recommendation based on the anatomic data for the patient, the comorbidity data for the patient, and based on an implant type of a prosthesis to be implanted in the patient during the shoulder replacement surgery.

Aspect 4: The method of aspect 3, wherein the implant type of the prosthesis is one of a glenoid cup implant or a glenosphere implant.

Aspect 5: The method of any of aspects 1-4, wherein the anatomic data for the patient include data describing soft tissue of a shoulder joint of the patient.

Aspect 6: The method of any of aspects 1-5, wherein the anatomic data for the patient include 3-dimensional anatomic measurements of a scapula and humerus of the patient.

Aspect 7: The method of any of aspects 1-6, wherein the anatomic data for the patient include one or more of: a critical shoulder angle, a distance from a humeral head center to a glenoid center, a distance from the acromion to the humeral head, a scapula critical shoulder sagittal angle, a glenoid coracoid process angle, an infraglenoid tubrical angle, a scapula acromion index, a humerus orientation, a humerus direction, a measure of humerus subluxation, or a humeral head best fit sphere.

Aspect 8: The method of aspect 7, wherein the anatomic data for the patient include any combination of two or more of: a critical shoulder angle, a distance from a humeral head center to a glenoid center, a distance from the acromion to the humeral head, a scapula critical shoulder sagittal angle, a glenoid coracoid process angle, an infraglenoid tubrical angle, a scapula acromion index, a humerus orientation, a humerus direction, a measure of humerus subluxation, or a humeral head best fit sphere.

Aspect 9: The method of any of aspects 1-8, wherein the anatomic data for the patient include data regarding bone loss of the patient.

Aspect 10: The method of any of aspects 1-9, wherein the anatomic data for the patient include data regarding bone quality of the patient.

Aspect 11: The method of any of aspects 1-10, wherein the anatomic data for the patient include data regarding osteophytes.

Aspect 12: The method of any of aspects 1-11, wherein the comorbidities of the patient include one or more of history of smoking, hypertension, chronic obstructive pulmonary disease (COPD), obesity, diabetes, steroid use, or heart failure.

Aspect 13: The method of any of aspects 1-12, wherein generating the recommendation comprises generating, by the computing system, the recommendation based on the anatomic data for the patient, the comorbidity data for the patient, and based on patient demographic information of the patient.

Aspect 14: The method of aspect 13, wherein the demographic information includes one or more of age, sex, weight, or height of the patient.

Aspect 15: The method of any of aspects 1-14, wherein generating the recommendation comprises applying, by the computing system, a machine learning (ML) model to the anatomic data for the patient and the comorbidity data for the patient to generate the recommendation.

Aspect 16: The method of aspect 15, wherein the ML model is an artificial neural network.

Aspect 17: The method of any of aspects 15-16, wherein the method further comprises training the ML model based on a plurality of training data sets, wherein for each respective training data set of the training data sets, the respective training data set specifies anatomic data for a respective earlier patient and comorbidity data for the respective earlier patient, and the respective training data set specifies whether the respective earlier patient underwent the surgery as the inpatient procedure or the outpatient procedure.

Aspect 18: The method of any of aspects 1-17, further includes generating, by the computing system, based on the anatomic data for the patient and the comorbidity data for the patient, a duration of stay estimate for the patient, wherein the duration of stay estimate for the patient is an estimate of a length of time the patient will stay in a healthcare facility after undergoing the surgery.

Aspect 19: The method of aspect 18, wherein: generating the recommendation comprises applying, by the computing system, a ML model to the anatomic data for the patient and the comorbidity data for the patient to generate the recommendation, and generating the duration of stay estimate comprises applying, by the computing system, the same ML model to the anatomic data for the patient and the comorbidity data for the patient to generate the duration of stay estimate for the patient.

Aspect 20: A computing system includes a memory configured to store anatomic data for a patient and comorbidity data for the patient; and one or more processing circuits configured to: generate, based on the anatomic data for the patient and the comorbidity data for the patient, a recommendation regarding whether the patient should undergo a surgery as inpatient procedure or an outpatient procedure; and output the recommendation.

Aspect 21: The computing system of aspect 20, wherein the one or more processing circuits are configured to perform the methods of any of aspects 2-19.

Aspect 22: A computing system includes means for storing anatomic data for a patient and comorbidity data for the patient; means for generating, based on the anatomic data for the patient and the comorbidity data for the patient, a recommendation regarding whether the patient should undergo a surgery as an inpatient procedure or an outpatient procedure; and means for outputting the recommendation.

Aspect 23: The computing system of aspect 19, further comprising means for performing the methods of any of aspects 2-19.

Aspect 24: A computer-readable medium having instructions stored thereon that, when executed, cause a computing system to perform the methods of any of aspects 1-19.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention. Moreover, techniques of this disclosure have generally been described with respect to human anatomy. However, the techniques of this disclosure may also be applied to animal anatomy in veterinary cases.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuitry,” as used herein may refer to any of the foregoing structures or any other structure suitable for implementation of the techniques described herein.

1: A computer-implemented method comprising:
obtaining, by a computing system, anatomic data for a patient and comorbidity data for the patient;
generating, by the computing system, based on the anatomic data for the patient and the comorbidity data for the patient, a recommendation regarding whether the patient should undergo a surgery as an inpatient procedure or an outpatient procedure; and
outputting, by the computing system, the recommendation.

2. (canceled)

3: The method of claim 1, wherein generating the recommendation comprises generating, by the computing system, the recommendation based on the anatomic data for the patient, the comorbidity data for the patient, and based on an implant type of a prosthesis to be implanted in the patient during the shoulder replacement surgery.

4: The method of claim 3, wherein;
the implant type of the prosthesis is one of a glenoid cup implant or a glenosphere implant.

5: The method of claim 1, wherein the anatomic data for the patient include data describing soft tissue of a shoulder joint of the patient.

6: The method of claim 1, wherein the anatomic data for the patient include 3-dimensional anatomic measurements of a scapula and humerus of the patient.

7: The method of claim 1, wherein the anatomic data for the patient include one or more of:
a critical shoulder angle,
a distance from a humeral head center to a glenoid center,
a distance from an acromion to the humeral head,
a scapula critical shoulder sagittal angle,
a glenoid coracoid process angle,
an infraglenoid tubrical angle,
a scapula acromion index,
a humerus orientation,
a humerus direction,
a measure of humerus subluxation, or
a humeral head best fit sphere.

8: The method of claim 7, wherein the anatomic data for the patient include any combination of two or more of:
a critical shoulder angle,
a distance from a humeral head center to a glenoid center,
a distance from an acromion to the humeral head,
a scapula critical shoulder sagittal angle,
a glenoid coracoid process angle,
an infraglenoid tubrical angle,
a scapula acromion index,
a humerus orientation,
a humerus direction,
a measure of humerus subluxation, or
a humeral head best fit sphere.

9: The method of claim 1, wherein the anatomic data for the patient include data regarding bone loss of the patient.

10: The method of claim 1, wherein the anatomic data for the patient include data regarding bone quality of the patient.

11: The method of claim 1, wherein the anatomic data for the patient include data regarding osteophytes.

12: The method of claim 1, wherein the comorbidities of the patient include one or more of history of smoking, hypertension, chronic obstructive pulmonary disease (COPD), obesity, diabetes, steroid use, or heart failure.

13: The method of claim 1, wherein generating the recommendation comprises generating, by the computing system, the recommendation based on the anatomic data for the patient, the comorbidity data for the patient, and based on patient demographic information of the patient.

14: The method of claim 13, wherein the demographic information includes one or more of age, sex, weight, or height of the patient.

15: The method of claim 1, wherein generating the recommendation comprises applying, by the computing system, a machine learning (ML) model to the anatomic data for the patient and the comorbidity data for the patient to generate the recommendation.

16: The method of claim 15, wherein;
the ML model is an artificial neural network,
the input data including the anatomic data and the comorbidity data for the patient,
generating the recommendation comprises:
providing numerical representations of the input data to individual neurons of an input layer of the artificial neural network; and
performing a forward propagation pass through the artificial neural network to generate one or more output values that include the recommendation.

17: The method of claim 15, wherein the method further comprises training the ML model based on a plurality of training data sets, wherein for each respective training data set of the training data sets, the respective training data set specifies anatomic data for a respective earlier patient and comorbidity data for the respective earlier patient, and the respective training data set specifies whether the respective earlier patient underwent the surgery as the inpatient procedure or the outpatient procedure.

18: The method of claim 1, further comprising:
generating, by the computing system, based on the anatomic data for the patient and the comorbidity data for the patient, a duration of stay estimate for the patient, wherein the duration of stay estimate for the patient is an estimate of a length of time the patient will stay in a healthcare facility after undergoing the surgery.

19: The method of claim 18, wherein:
generating the recommendation comprises applying, by the computing system, a ML model to the anatomic data for the patient and the comorbidity data for the patient to generate the recommendation, and
generating the duration of stay estimate comprises applying, by the computing system, the same ML model to the anatomic data for the patient and the comorbidity data for the patient to generate the duration of stay estimate for the patient.

20: A computing system comprising:
a memory configured to store anatomic data for a patient and comorbidity data for the patient; and
one or more processing circuits configured to:
generate, based on the anatomic data for the patient and the comorbidity data for the patient, a recommendation regarding whether the patient should undergo a surgery as inpatient procedure or an outpatient procedure; and
output the recommendation.

21-23. (canceled)

24: A non-transitory computer-readable medium having instructions stored thereon that, when executed, cause a computing system to:
obtain anatomic data for a patient and comorbidity data for the patient;
generate, based on the anatomic data for the patient and the comorbidity data for the patient, a recommendation regarding whether the patient should undergo a surgery as an inpatient procedure or an outpatient procedure; and
output the recommendation.
